SEQUENCE LISTING (7 sequences)

SEQ ID NO: 1 - DNA, 33 bp, Artificial Sequence
  Probe
tttctgttgcttattgctgttactttttcagta

SEQ ID NO: 2 - DNA, 22 bp, Artificial Sequence
  Probe
atcctggacgatcaacttatgc

SEQ ID NO: 3 - DNA, 22 bp, Artificial Sequence
  Probe
gagggcgagaatcttcaataat

SEQ ID NO: 4 - DNA, 23 bp, Artificial Sequence
  Probe
ggagggcgagaatcttcaataat

SEQ ID NO: 5 - DNA, 27 bp, Artificial Sequence
  Probe
tattgttagcatacagaagtctccaat

SEQ ID NO: 6 - DNA, 23 bp, Artificial Sequence
  Probe
ggagggcgagaatcttcaataat

SEQ ID NO: 7 - DNA, 27 bp, Artificial Sequence
  Probe
tattgttagcatacagaagtctccaat

(19) United States
(12) Patent Application Publication
(10) Pub. No.: US 2014/0325714 A1
(43) Pub. Date: Oct. 30, 2014

(54) Mutant Soybean Line

(71) Applicant: The United States of America, as Represented by the Secretary of Agriculture, Washington, DC (US)

(72) Inventors: Jason D. Gillman, Columbia, MO (US); Ivan R. Baxter, St. Louis, MO (US); Kristin D. Bilyeu, Columbia, MO (US)

(21) Appl. No.: 14/200,581

(22) Filed: Mar. 7, 2014

Related U.S. Application Data
(60) Provisional application No. 61/782,875, filed on Mar. 14, 2013.

(51) Int. Cl. A01H 5/10

(52) U.S. Cl. CPC A01H 5/10
USPC 800/312; 800/298; 435/410; 435/415

(57) ABSTRACT

Seed phytate is a repository of phosphorus and minerals in soybean seeds which limits phosphorus and mineral bioavailability for monogastric animals (e.g. humans, swine and poultry) due to insufficient digestive tract phytase activity. A soybean line containing two recessive mutations produces viable seeds that contain surprising reductions in total seed phytate and significantly higher levels of inorganic phosphate compared to normal soybeans.

### CROSS REFERENCE TO RELATED APPLICATIONS:

This application claims priority to U.S. Patent Application 61/782,875 filed Mar. 14, 2013.

### BACKGROUND OF INVENTION

1. Field of Invention

This invention relates a soybean line containing two recessive mutations that result in the production of seeds containing surprisingly low amounts of phytate and surprising high amounts of inorganic phosphate, compared to both soybean plants containing each mutation separately and to wild-type soybean plants.

2. Prior Art Description

Phytic acid (myo-inositol-1,2,3,4,5,6-hexa-kisphosphate) is a storage form of phosphorus in plant seeds. Monogastric animals (including humans, poultry and swine) lack sufficient levels of phytase to utilize this phosphorus (Raboy, et al., Seed phosphorus and the development of low-phytate crops, pp. 111-132 in: Inositol Phosphates: Linking Agriculture and Environment (Turner, et al., eds.) CAB International, Oxfordshire, UK (2007b)). In addition, the highly negative overall charge of phytic acid causes it to spontaneously precipitate cationic species, including a number of critical micronutrients such as Fe²⁺ and Zn²⁺ (Raboy, V., The Journal of Nutrition, 132:503S-505S (2002)). Phosphorus is an essential animal macronutrient, and feedstocks for non-ruminants are routinely supplemented with nonsustainable inorganic rock phosphate for optimal growth and weight gain (Waldroup, et al., Poultry Science 79:1451-1459 (2000)). In addition to slightly increasing the cost of feed mixtures, excess phosphate can accumulate in the environment because manure is used as a fertilizer (Raboy (2002); Raboy, et al. (2007b)). Unutilized phosphorus has the potential to accelerate eutrophication when released into fresh water streams and lakes (Correll, D., J. Environ. Qual. 27:261-266 (1998); Raboy, V., Nature Biotechnology 25:874-875 (2007a)). In soybean, phytic acid typically comprises the majority of the total seed phosphorus (approximately 72%) (Raboy, et al., Crop Science 24:431-434 (1984)), although phytic acid content may vary based on the nutritional status of the maternal plant. The use of seeds with lowered phytic acid in animal feed mixtures has the potential to reduce, or potentially eliminate, the need for supplementation with exogenous inorganic phosphate (Raboy, et al. (2007b)), as well as increasing the bioavailability of critical micronutrients (Zhou, et al., The Journal of Nutrition 122:2466-2473 (1992)).

Thus there is a great deal of interest in both the applied goal of reducing the level of phytic acid accumulation in seeds as well as elucidating the biosynthetic pathways for phytic acid production. In soybean, mutagenesis was used to develop several independent mutant lines (and, in one case, a spontaneously occurring mutant) with significant reductions in phytic acid content and a concomitant increase in bio-available inorganic phosphate (Hitz, et al., Plant Physiology 128:650-660 (2002); U.S. Pat. No. 8,003,856; Wilcox, et al., Crop Science 40:1601-1605 (2000)). Mutations affecting two different classes of genes were identified as causative: 1) a myo-inositol phosphate synthase (MIPS) gene (Hitz, et al. (2002); U.S. Pat. No. 8,003,856; Yuan, et al., TAG Theoretical and Applied Genetics 115:945-957 (2007)); and 2) two phytic acid specific ATP-Binding Cassette transporter paralogs, which have been named low phytic acid (Lpa) genes (Gillman, et al., Plant Genome 2:179:190 (2009); U.S. Pat. No. 8,003,856; Shi, et al., Nature Biotechnology 25:930-7 (2007); Nagy, et al., J. of Biolog. Chem. 284:33614-33622 (2009)). Deleterious impacts on germination/field emergence was observed as consequences of the presence of either of these two biochemically and genetically distinct sources of low phytic acid soybean (Anderson and Fehr, Crop Science 48:929-932 (2008); Maupin and Rainey, Crop Science 51:1946-1955 (2011); Meis, et al., Crop Sciences 43:1336-1339 (2003)). Unfortunately, the variable environmental effect has so far precluded a clear understanding of the physiological basis of the germination defect, aside from being associated with the presence of mutant alleles.

Mutations in a single MIPS gene result in modest reductions in phytic acid content as compared to wild type lines, as well an additional beneficial reduction in indigestible raffinose family oligosaccharides (Hitz, et al. (2002)). As such, mutations affecting MIPS would be the more logical a priori target for breeding efforts. However, MIPS mutant lines have higher levels of the anti-nutritional compound phytic acid, less bioavailable phosphorus and feature more significant germination defects as compared to Lpa mutant lines (Maupin and Rainey (2011)). Indeed, complete silencing of the MIPS gene has been shown to be embryo lethal in soybean (Nunes, et al., Planta 224:125-132 (2006)).

In contrast, silencing the soybean Lpa genes failed to result in either embryo or seedling lethality in a large number of transgenic events in maize and soybean (Shi, et al. (2007)), and the germination issues appear to have a lower overall effect (Maupin and Rainey (2011)) which can, at least partially, be ameliorated by appropriate genetic selection (Anderson and Fehr (2008); Spear and Fehr, Crop Science 47:1354-1360 (2007)). Thus, there is a goal to utilize Lpa gene mutations to develop a crop having seeds with low phytic acid and high inorganic phosphorus levels compared to both wild-type crop seeds and other low phytic acid crop seeds.

### SUMMARY OF THE INVENTION

It is an object of this invention to have a plant containing the alleles lpa1-a and lpa2-b.

It is an object of this invention to have a plant containing the alleles lpa1-a and lpa2-b. It is a further object of this invention that the plant of this invention produces seeds which have reduced phytate or phytic acid levels and higher inorganic phosphate levels compared to the seeds of the wild-type version of this plant.

It is an object of this invention to have a plant containing the alleles lpa1-a and lpa2-b. It is another object of this invention that the plant is Glycine max. It is a further object of this invention to have a Glycine max that produces seeds that contain lower levels of phytate or phytic acid and higher levels of inorganic phosphate compared to the seeds of wild-type Glycine max.

It is an object of this invention to have seeds of a plant where the seeds contain the alleles lpa1-a and lpa2-b.

It is an object of this invention to have seeds of a plant where the seeds contain the alleles lpa1-a and lpa2-b. It is a further object of this invention that the seeds contain lower levels of phytate or phytic acid and higher levels of inorganic phosphate compared to the seeds of the wild-type version of this plant. It is another object of this invention that the plant is Glycine max.

It is an object of this invention to have a cell of a plant where the cell contains the alleles lpa1-a and lpa2-b.

It is an object of this invention to have a cell of a plant where the cell contains the alleles lpa1-a and lpa2-b. It is a further object of this invention that the seeds of this plant contain lower levels of phytate or phytic acid and higher levels of inorganic phosphate compared to the seeds of the wild-type version of this plant. It is another object of this invention that the plant is Glycine max.

It is an object of this invention to have germplasm of a plant where the germplasm contains the alleles lpa1-a and lpa2-b.

It is an object of this invention to have germplasm of a plant which will yield seeds that contain the alleles lpa1-a and lpa2-b.

It is an object of this invention to have germplasm of a plant which will yield seeds that contain the alleles lpa1-a and lpa2-b. It is a further object of this invention that the seeds of this plant contain lower levels of phytate or phytic acid and higher levels of inorganic phosphate compared to the seeds of the wild-type version of this plant. It is another object of this invention that the plant is Glycine max.

It is an object of this invention to have a plant which contains mutant alleles that affect Lpa1 and Lpa2 genes.

It is an object of this invention to have a plant which contains mutant alleles that affect Lpa1 and Lpa2 genes. It is another object of this invention that the seeds of this plant contain lower levels of phytate or phytic acid and higher levels of inorganic phosphate compared to the seeds of a plant containing the lpa1-a and lpa2-a mutant alleles.

It is an object of this invention to have a plant which contains mutant alleles that affect Lpa1 and Lpa2 genes. It is a further object of this invention that the plant be a mutant Glycine max. It is another object of this invention that the seeds of this mutant Glycine max contain lower levels of phytate or phytic acid and higher levels of inorganic phosphate compared to the seeds of a mutant Glycine max containing the lpa1-a and lpa2-a mutant alleles.

It is an object of this invention to have seeds of a plant where the seeds contain mutant alleles that affect Lpa1 and Lpa2 genes.

It is an object of this invention to have seeds of a plant where the seeds contain mutant alleles that affect Lpa1 and Lpa2 genes. It is another object of this invention that the seeds contain lower levels of phytate or phytic acid and higher levels of inorganic phosphate compared to the seeds of a plant containing the lpa1-a and lpa2-a mutant alleles.

It is an object of this invention to have seeds of a mutant Glycine max where the seeds contain mutant alleles that affect Lpa1 and Lpa2 genes. It is another object of this invention that the seeds contain lower levels of phytate or phytic acid and higher levels of inorganic phosphate compared to the seeds of a mutant Glycine max containing the lpa1-a and lpa2-a mutant alleles.

It is an object of this invention to have germplasm of seeds of a plant where the seeds contain mutant alleles that affect Lpa1 and Lpa2 genes. It is another object of this invention that the seeds contain lower levels of phytate or phytic acid and higher levels of inorganic phosphate compared to the seeds of a plant containing the lpa1-a and lpa2-a mutant alleles. It is further object of this invention that the plants being mutant Glycine max.

It is an object of this invention to have a cell of a plant where the seeds of the plant contain mutant alleles that affect Lpa1 and Lpa2 genes.

It is an object of this invention to have a cell of a plant where the seeds of the plant contain mutant alleles that affect Lpa1 and Lpa2 genes. It is another object of this invention that the seeds contain lower levels of phytate or phytic acid and higher levels of inorganic phosphate compared to the seeds of a plant containing the lpa1-a and lpa2-a mutant alleles. It is further object of this invention that the plants being mutant Glycine max.

It is an object of this invention to have a seed of a soybean plant containing two non-lethal mutations such that the seed has a phenotype characterized by having lower levels of phytate or phytic acid and higher levels of inorganic phosphate compared to the seeds of soybean plant cultivar TN09-239.

It is an object of this invention to have a seed of a soybean plant containing two non-lethal mutations such that the seed has a phenotype characterized by having lower levels of phytate or phytic acid and higher levels of inorganic phosphate compared to the seeds of soybean plant cultivar TN09-239. It is a further object of this invention that the phenotype is caused by mutations in Lpa1 and Lpa2 genes.

It is a further object of this invention to have a soybean plant that is produced by a seed which contains two non-lethal mutations such that the seed has a phenotype characterized by having lower levels of phytate or phytic acid and higher levels of inorganic phosphate compared to the seeds of soybean plant cultivar TN09-239.

It is yet a further object of this invention to have a soybean plant that is produced by a seed which contains two non-lethal mutations such that the seed has a phenotype characterized by having lower levels of phytate or phytic acid and higher levels of inorganic phosphate compared to the seeds of soybean plant cultivar TN09-239. It is another object of this invention that the phenotype is caused by mutations in Lpa1 and Lpa2 genes.

It is another object of this invention to have germplasm that will yield a soybean seed which contains two non-lethal mutations such that the seed has a phenotype characterized by having lower levels of phytate or phytic acid and higher levels of inorganic phosphate compared to the seeds of soybean plant cultivar TN09-239.

It is another object of this invention to have germplasm that will yield a soybean seed which contains two non-lethal mutations such that the seed has a phenotype characterized by having lower levels of phytate or phytic acid and higher levels of inorganic phosphate compared to the seeds of soybean plant cultivar TN09-239. It is another object of this invention that the phenotype is caused by mutations in Lpa1 and Lpa2 genes.

### BRIEF DESCRIPTION OF THE DRAWINGS

FIG. 1A is a representation of the mutant alleles of LPA2 and the Lpa2 gene, with exons indicated by dark boxes, introns represented by lines. The predicted transcriptional start site is indicated by arrow. The position of codon 1039 which is affected in both low phytic acid mutant lines CX1834/M153 (missense, light line) and M766 (nonsense, asterisk) is indicated by an arrow. FIG. 1B is the sequencing traces for a region surrounding codon 1039 in LPA2. In lines bearing the CX1834/M153 allele (lpa2-a) a missense mutation occurs (R1039K), and in lines bearing the M766 derived allele (lpa2-b) a nonsense mutation has occurred (R1039STOP).

FIG. 2 shows histograms displaying mean phosphorus phenotypes of F₂ genotypic classes from the M766 by ‘Williams 82’ cross and comparison to parental lines. The mean of the genotypic class for total seed phosphorus (P) is indicated by the total height of the histogram, inorganic phosphate (Pi) is indicated by light gray. One standard deviation from the mean is indicated by brackets. The nine possible genotypic classes for LPA1/lpa1 and LPA2/lpa2 of F₂ progeny from M766×‘Williams 82’ are indicated along with their number of samples (n=), and parental and control lines grown in the same growth chamber are included for reference. The allelic status of the lpa1 and lpa2 genes follows the convention: “T” indicates a functional allele derived from ‘Williams 82’, and “b” indicates the mutant allele derived from M766, with the bold font representing the null allele of lpa2-b from M766.

FIG. 3 shows histograms displaying phosphorus phenotypes of F_{2:3} selections from the M766 by ‘Williams 82’ cross and comparison to parental lines. Each histogram represents the mean of the genotypic class, though each seed is individually ground and subsampled for DNA isolation/genotyping and phosphorus assays. Total seed phosphorus (P) is indicated by the total height of the histogram, inorganic phosphate (Pi) is indicated by light gray, phytic acid (PA) is indicated by dark gray, and the difference between total P minus PA and Pi (cellular P) is indicated in gray. Parental lines grown in the same environment are included for reference.

FIG. 4 shows histograms displaying mean phosphorus phenotypes of F₂ genotypic classes from the M766 by TN09-239 cross and comparison to parental lines. The mean of the genotypic class for total seed phosphorus is indicated by the total height of the histogram, inorganic phosphate (P) is indicated by light gray. One standard deviation from the mean is indicated by brackets. The allelic status of the lpa1 and lpa2 genes follows the convention: “a” indicates a mutant allele derived from TN09-239, and “b” indicates the mutant allele derived from M766, with the bold font representing the null alleles of lpa1-a from TN09-239 and lpa2-b from M766. The nine possible genotypic classes for lpa1-a/lpa1-b and lpa2-allpa2-b of F₂ progeny from M766×TN09239 are indicated along with their number of samples (n=), and parental and control lines grown in the same growth chamber are included for reference.

FIG. 5 shows histograms displaying phosphorus phenotypes of F_{2:3} selections from the M766 by TN09-239 cross and comparison to parental lines. Each histogram represents the mean of the genotypic class, though each seed is individually ground and subsampled for DNA isolation/genotyping and phosphorus assays. Total seed phosphorus (P) is indicated by the total height of the histogram, inorganic phosphate (Pi) is indicated by light gray, phytic acid (PA) is indicated by dark gray, and the difference between total P minus PA and Pi (cellular P) is indicated in gray. The first four genotypic classes represent F_{2:3} progeny from M766×TN09-239 selections for all four possible homozygous genotypic combinations of lpa1 and lpa2 mutant alleles. The next three histograms are from progeny of a single F₂ plant from a cross between M766×TN09-239 that was fixed for lpa1-a. Parental and control lines grown in the same environment are included for reference.

FIG. 6A is a cartoon depiction of the Lpa1 or lpa1-b locus intron SNP (T>A 7 basepairs upstream of exon 10) and mRNA consequences of the presence of the intron mutation. Exons are indicated by dark boxes, introns represented by lines. The predicted transcriptional start site is indicated by arrow, and the placement of the intron mutation is indicated by a vertical arrow. Primers used for RT-PCR are indicated by black arrows located above exon 9 and exon 10. FIG. 6B are the sequence traces from RT-PCR products amplified using gene specific primers located in exon 9 and exon 10.

FIG. 7 shows the phosphorus phenotypic data for all nine possible F₂ genotypic classes, as determined by genotyping assays and phenotyping on individual F₂ seeds from crosses between M766×TN09-239 or M766×W82.

FIG. 8 shows the seed phosphorus phenotypic data for F_{2:3} genotypic selections from crosses between M766×TN09-239 or M766×W82.

### DETAILED DESCRIPTION OF THE INVENTION

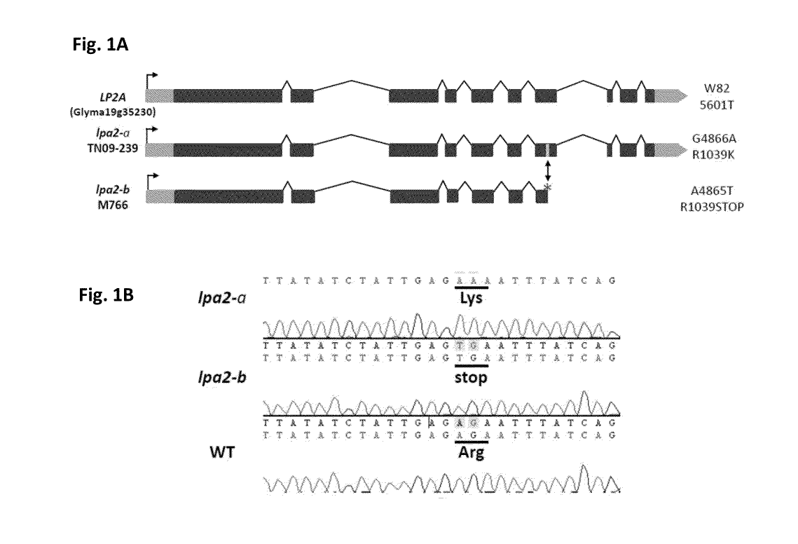

The ethyl methanesulfonate induced mutant soybean line M153 (Wilcox, et al. (2000)) was used to develop a partially adapted breeding line CX1834 (Oltmans, et al., Crop Science 44:433-435 (2004)). A nonsense mutation in one Lpa paralog (Glyma03g32500:lpa1-a) and a missense R1039K substitution affecting an ancestrally invariant residue within the second paralog (Glyma19g35230:lpa2-a) in the soybean line M153 was identified and reported (Gillman, et al. (2009)). (It is noted that for the sake of clarity, nonsense mutations, such as “lpa1-a”, are indicated by presence of bold type.) Genetic analysis revealed a rational biochemical basis for the duplicate dominant epistatic interaction, based on the combination of two independent deleterious mutations (one nonsense and one missense) in two of the genes encoding Lpa paralogous genes (lpa1-a and lpa2-a) (Gillman, et al. (2009)). These genes were sequenced in a “sister” low phytic acid, ethyl methanesulfonate induced mutant soybean line, M766, created in the same mutagenesis experiment as soybean lines M153/CX1834 (Wilcox, et al. (2000)). The M766 version of the Glyma03g32500 Lpa1 gene contains a single SNP in intron 9 compared to the ‘Williams 82’ reference sequence, and is designated herein as lpa1-b (Gillman, et al. (2009)). Soybean mutant line M766 bears a nonsense mutation in Glyma19g35230 (lpa2-b), which coincidentally affects the exact same codon (R1039*, see FIG. 1A) as the missense mutation in M153. Thus the current collection of variant Lpa alleles is: two null alleles, one of lpa1-a from M153 and one of lpa2-b from M766; one missense allele of lpa2-a from M153; and one lpa1-b allele from M766 that does not differ in the coding sequence from the reference ‘Williams 82’ allele, but that contains a single SNP within intron 9 of the gene.

There remains a need for a soybean line that expresses low phytate and high inorganic phosphate levels within the beans as compared to wild-type soybean plants and as compared to prior art low phytic acid soybean plants. This invention involves the generation of such a soybean line. This mutant soybean line is created by the crossing of low phytate line TN09-239 (which possesses a nonsense lpa1-a mutation and a missense lpa2-a mutation originally derived from M153) (obtained from Vince Pantalone at the University of Tennessee, Knoxville, Tenn.)×M766 (which contains the unique polymorphic allele lpa1-b and a nonsense lpa2-b mutations) (obtained from USDA) and analyzing the F₂ and F₃ progeny. While it was previously suggested (Gillman, et al. (2009)) that such a cross would be useful to examine, a soybean line with unexpectedly low phytate levels and unexpectedly high inorganic phosphate levels in the beans as compared to wild-type soybeans and to other low phytate soybeans is obtained. This soybean line contains the nonsense lpa1-a mutation and the nonsense lpa2-b mutation. Because this mutant soybean line possesses surprisingly increased phosphate levels available for absorption by monogastric animals, it has an even greater than expected enhanced nutritional value for food and feed.

The DNA sequence of Lpa2, lpa2-a, lpa2-b, and lpa1-a are provided in Gillman, et al. (2009). The DNA sequence of lpa1-b is provided in Gillman, et al., The Plant Genome doi: 10.3835/plantgenome2012.06.0010 (Dec. 12, 2012).

While this invention describes a mutant soybean line having low levels of phytic acid and high levels of inorganic phosphorus in the beans (or seeds) compared to wild-type soybean lines and that the reason for this phenotype are the presence of two mutated genes, lpa1-a and lpa2-b. Using ethyl methanesulfonate or another mutagenic compound, one can generate mutations in the Lpa genes of soybean plants. Using the assays described herein one can screen the mutated crop plants for the mutant alleles lpa-1b and lpa-2a and cross the plants to generate plants containing both alleles. Such crop plants will have similar characteristics as the soybean plants in the present invention, reduced phytate levels compared to wild-type crop plants and to other low phytate levels crop plants and elevated levels of inorganic phosphate levels compared to wild-type crop plants and to other high inorganic phosphate levels crop plants.

As described herein, the mutant alleles lpa1-a and lpa2-b are nonsense mutations that result in substitution of a “STOP translation” codon in place of a normal amino acid. The lpa1-a and lpa2-b mutations result in truncated protein products which have altered functionality. Mutations within both alleles could be created, and those mutations which generate truncated protein products having similar activity as the protein products of lpa1-a and lpa2-b can be identified. Such mutations could result in increased inorganic phosphate and decreased phytic acid identical (or very similar) to the effect of the lpa1-a and lpa2-b mutations.

The term “nucleic acid” as used herein, refers to a polymer of ribonucleotides or deoxyribonucleotides. Typically, “nucleic acid” polymers occur in either single- or double-stranded form, but are also known to form structures comprising three or more strands. The term “nucleic acid” includes naturally occurring nucleic acid polymers as well as nucleic acids comprising known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examplary analogs include, without limitation, phosphorothioates, phosphoramidates, methyl phosphonates, chiral-methyl phosphonates, 2-O-methyl ribonucleotides, peptide-nucleic acids (PNAs). “DNA”, “RNA”, “polynucleotides”, “polynucleotide sequence”, “oligonucleotide”, “nucleotide”, “nucleic acid”, “nucleic acid molecule”, “nucleic acid sequence”, “nucleic acid fragment”, and “isolated nucleic acid fragment” are used interchangeably herein.

The term “label” as used herein, refers to a composition detectable by spectroscopic, photochemical, biochemical, immunochemical, or chemical means. Exemplary labels include ³²P, fluorescent dyes, electron-dense reagents, enzymes (e.g., as commonly used in an ELISA), biotin, digoxigenin, or haptens and proteins for which antisera or monoclonal antibodies are available.

As used herein a nucleic acid “probe”, oligonucleotide “probe”, or simply a “probe” refers to a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (e.g., 7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. In one embodiment, probes are directly labeled as with isotopes, chromophores, lumiphores, chromogens, etc. In another embodiment probes are indirectly labeled e.g., with biotin to which a streptavidin complex may later bind. By assaying for the presence or absence of the probe, one can detect the presence or absence of the select sequence or subsequence. Thus, a probe is set of polynucleotides that can bind, either covalently, through a linker or a chemical bond, or noncovalently, through ionic, van der Waals, electrostatic, or hydrogen bonds, to a label such that the presence of the probe may be detected by detecting the presence of the label bound to the probe.

The term “primer” as used herein, refers to short nucleic acids, typically a DNA oligonucleotide of at least about 15 nucleotides in length. In one embodiment, primers are annealed to a complementary target DNA strand by nucleic acid hybridization to form a hybrid between the primer and the target DNA strand. Annealed primers are then extended along the target DNA strand by a DNA polymerase enzyme. Primer pairs can be used for amplification of a nucleic acid sequence, e.g., by the polymerase chain reaction (PCR) or other nucleic-acid amplification methods known in the art.

PCR primer pairs are typically derived from a known sequence, for example, by using computer programs intended for that purpose such as Primer (Version 0.5©1991, Whitehead Institute for Biomedical Research, Cambridge, Mass.). One of ordinary skill in the art will appreciate that the specificity of a particular probe or primer increases with its length. Thus, for example, a primer comprising 20 consecutive nucleotides of a promoter complex sequence will anneal to a particular sequence with a higher specificity than a corresponding primer of only 15 nucleotides. Thus, in one embodiment, greater specificity of a nucleic acid primer or probe is attained with probes and primers selected to comprise 20, 25, 30, 35, 40, 50 or more consecutive nucleotides of a selected sequence.

Oligonucleotides and polynucleotides that are not commercially available can be chemically synthesized e.g., according to the solid phase phosphoramidite triester method first described by Beaucage and Caruthers, Tetrahedron Letts. 22:1859-1862 (1981), or using an automated synthesizer, as described in Van Devanter et al., Nucleic Acids Res. 12:6159-6168 (1984). Other methods for synthesizing oligonucleotides and polynucleotides are known in the art. Purification of oligonucleotides is by either native acrylamide gel electrophoresis or by anion-exchange HPLC as described in Pearson & Reanier, J. Chrom. 255:137-149 (1983).

Nucleic acid probes and primers are readily prepared based on the nucleic acid sequences disclosed herein. Methods for preparing and using probes and primers and for labeling and guidance in the choice of labels appropriate for various purposes are discussed, e.g., in Sambrook et al., Molecular Cloning, A Laboratory Manual 2nd ed. 1989, Cold Spring Harbor Laboratory; and Current Protocols in Molecular Biology, Ausubel et al., eds., 1994—current, John Wiley & Sons).

The phrase “selectively hybridizes to” or “specifically hybridizes to” refers to the binding, duplexing, or hybridizing of a molecule only to a particular nucleotide sequence under stringent hybridization conditions when that sequence is present in a complex mixture (e.g., total cellular or library DNA or RNA). In general, two nucleic acid sequences are said to be “substantially identical” when the two molecules or their complements selectively or specifically hybridize to each other under stringent conditions.

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength and pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C. However, other high stringency hybridization conditions known in the art can be used.

This invention utilizes routine techniques in the field of molecular biology. Basic texts disclosing the general methods of use in this invention include Sambrook et al., Molecular Cloning—A Laboratory Manual (2nd Ed.), Vol. 1-3, Cold Spring Harbor Laboratory, Cold Spring Harbor, N.Y. (1989); Kriegler, Gene Transfer and Expression: A Laboratory Manual (1990); and Ausubel et al. (eds.), Current Protocols in Molecular Biology (1994). Unless otherwise noted, technical terms are used according to conventional usage. Definitions of common terms in molecular biology maybe found in e.g., Benjamin Lewin, Genes IX, published by Oxford University Press (2007) (ISBN 0763740632); Krebs, et al. (eds.), The Encyclopedia of Molecular Biology, published by Blackwell Science Ltd. (1994) (ISBN 0-632-02182-9); and Robert A. Meyers (ed.), Molecular Biology and Biotechnology: a Comprehensive Desk Reference, published by VCH Publishers, Inc. (1995) (ISBN 1-56081-569-8).

Having now generally described this invention, the same will be better understood by reference to certain specific examples and the accompanying drawings, which are included herein only to further illustrate the invention and are not intended to limit the scope of the invention as defined by the claims. The examples and drawings describe at least one, but not all embodiments, of the inventions claimed. Indeed, these inventions may be embodied in many different forms and should not be construed as limited to the embodiments set forth herein; rather, these embodiments are provided so that this disclosure will satisfy applicable legal requirements.

### EXAMPLE 1

### Development of Soybean Populations Segregating for M766 Derived Alleles of LPA1 and LPA2 and Genotype Assessment

To investigate the roles of distinct alleles of the Lpa1 (Glyma03g32500) and Lpa2 (Glyma19g35230) genes in different combinations, two populations are developed by crossing, during the summer, the low phytic acid soybean line M766 (lpa1-b/lpa2-b) with the soybean reference cultivar ‘Williams 82’ (Bernard and Cremeens, Crop Science 28:1027-1028 (1988)) and also with the low phytic acid soybean line TN09-239 (lpa1-a/lpa2-a)(Gillman, et al. (2009)). ‘Williams 82’ possesses typical soybean levels of inorganic phosphate/phytic acid whereas TN09-239 (lpa1-a/lpa2-a) is a BC₄ low phytic acid soybean line developed by backcrossing CX18341-2 to 5601T (Pantalone, et al., Crop Science 43:1123-1124 (2003)). Crossing the low phytic acid soybean line M766 with ‘Williams 82’ a soybean line with wild-type levels of phytic acid, enables one to determine if the lpa1-b allele is linked to the low phytate phenotype. F₂ seed are produced in a growth chamber with a day/night cycle of 28° C. day for 13.5 hours, followed by 22° C. night for 10.5 hours. Nutrients are provided by Osmocote beads (Scotts Company, Marysville, Ohio). Putative F₁ plants are verified by lpa2-a/b assays as described in Gillman, et al. (2009).

Sequence differences between wild-type and mutant lpa genes permit tracking of the alleles in the two segregating populations of the soybean crosses. The M766 lpa1-b allele contains a unique single polymorphism when compared to the reference sequence for ‘Williams 82’, located within intron 9 (T₅₂₀₂A, relative to the start codon) (Gillman, et al. (2009)). This single polymorphism is used to develop the following Simpleprobe assay (Roche Applied Sciences, Indianapolis, Ind.) which is used according to manufacturer's instructions. The M766 intron T₅₂₀₂A SNP is detected through melting curve analysis to measure the disassociation kinetics of a Simpleprobe oligonucleotide (Fluorescein-SPC-TTTCTGTTGCTTATTGCTGTTACTTTTTCAGTA-Phosphate) (SEQ ID NO:1) which matches the ‘Williams 82’ Glyma03g32500/LPA1 sequence (http://www.phytozome.net/soybean). Asymmetric PCR using a 1:5 ratio of primers (forward primer 5′-ATCCTGGACGATCAACTTATGC-3′ (SEQ ID NO:2); reverse primer 5′-GAGGGCGAGAATCTTCAATAAT-3′ (SEQ ID NO:3)) provides template for Simpleprobe binding. Asymmetric PCR reactions and melting curve analysis are carried out as previously described in Gillman, et al. (2009) using a Lightcycler 480 II instrument (Roche, Basel, Switzerland).

A second Simpleprobe assay that correctly distinguishes wild-type Lpa2 alleles from both the missense and the nonsense lpa2 alleles (lpa2-a and lpa2-b, respectively), and is also able to distinguish the two mutant alleles from each other, is used to track the alleles in the progeny of the crosses (see Gillman, et al. (2009)). Further this second Simpleprobe assay is used to track lpa1-a segregation. These assays are performed using the SimpleProbe oligonucleotide probes purchased from Roche Applied Sciences (Indianapolis, Ind.) using either DNA isolated from seed tissue, or using leaf extract punches from FTA cards (Whatman, a division of GE Healthcare, Piscataway, N.J.) according to manufacturer's recommendations. The sequences of the probes, PCR parameters, and melting curve analyses are described in Gillman, et al. (2009). The lpa1-a, lpa2-a, and lpa2-b alleles were previously identified by crossing with CX1834 (see Gillman, et al. (2009)).

For a subset of the verified F₁ plants, the F₂ progeny seed from two plants (M766×‘Williams 82’, n=65) or one plant (M766×TN09-239, n=63) are harvested and are lyophilized, and individual seeds are ground with a mortar and pestle. The ground tissue is then analyzed using the above described assays and, as applicable, the assays described below.

Additional F₂ seed (from verified F₁ plants) are hand-planted during the next spring at the Bradford experimental field location to produce F₃ seed in a field environment. Leaf presses of the F₂ plants are made in the field with FTA cards (Whatman Ltd., a division of GE Healthcare, Piscataway, N.J.) for DNA isolation/genotyping assays as described above. Based on lpa1/lpa2 marker analysis, a subset of F₂ plants are selected for further analysis. Seed from F₂ or F₃ plants are harvested when pods are mature, and five F₃ seed are lyophilized, and individually ground with a mortar and pestle. Mature, lyophilized seed powder is subdivided and used for three distinct analyses: 1) DNA isolation for marker genotyping as described above and below; 2) Determination of free inorganic phosphate according to the protocol set forth in Gillman, et al. (2009); and Wilcox, et al. (2000) (also referred to as Chen's method) and described below; and 3) ICP-MS (inductively coupled plasma mass spectrometry) analysis to determine total phosphorus and other ionomic constituents as described below. F₃ seed is also analyzed for phytic acid content by HPLC (Chen and Li, J. Chromatogr. A 1018:41-52 (2003)) as described below. Wild-type and mutant alleles of Lpa1 and Lpa2 segregate in the ‘Williams 82’ cross, while only mutant alleles of lpa1 and lpa2 segregate when M766 is crossed with TN09-239 (see FIG. 1A and FIG. 1B).

### EXAMPLE 2

### Evaluation of Genetic Loci which Influence Seed Phosphate Concentration in M766

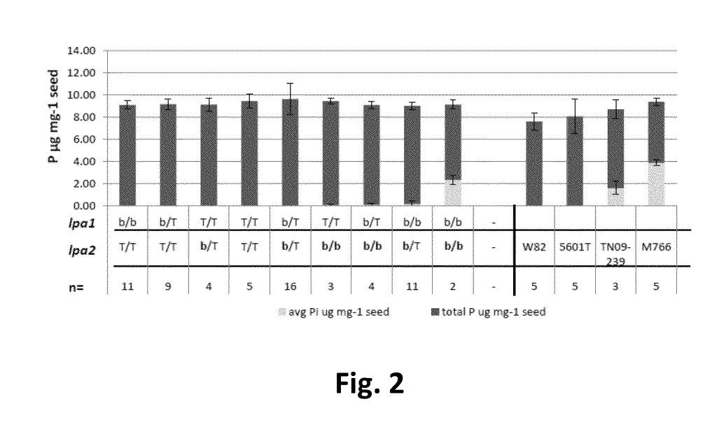

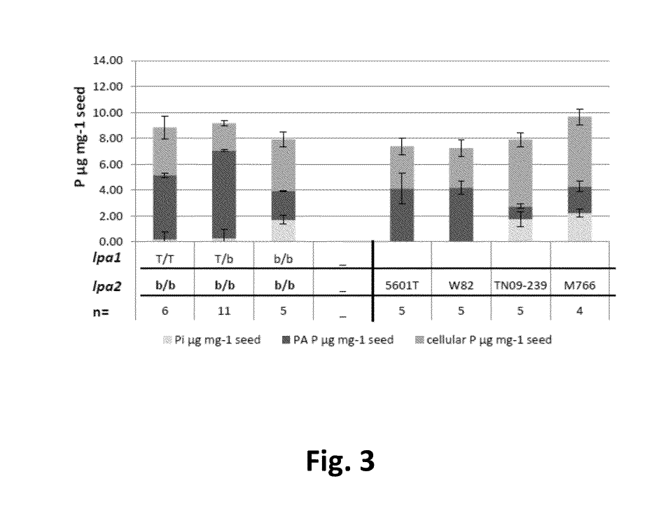

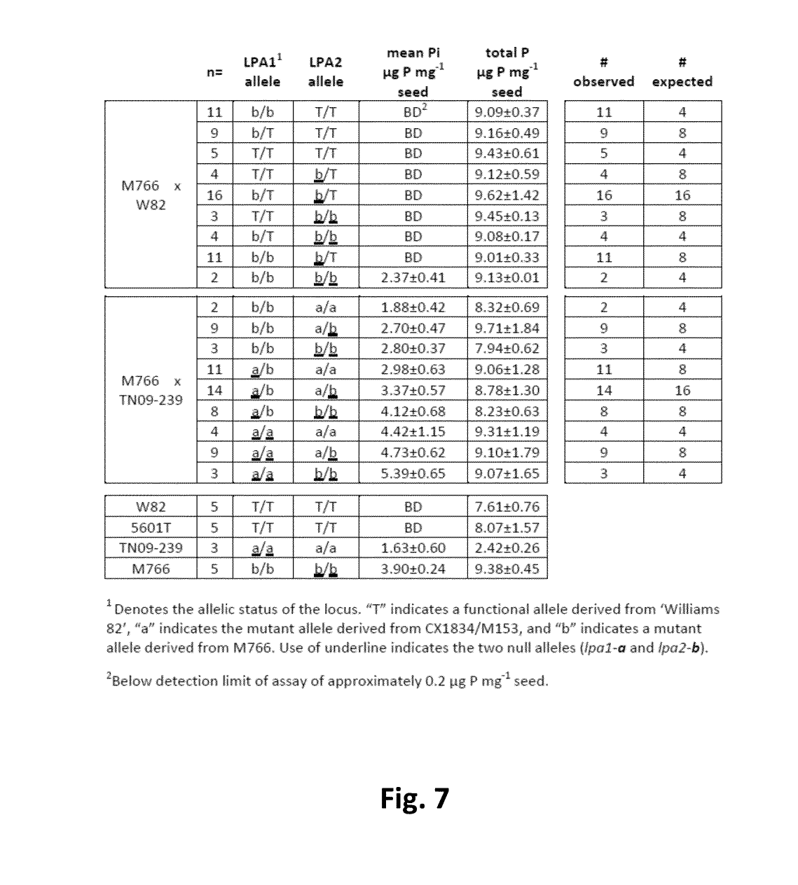

Based on previous results with M153 derived lines containing the lpa1-a and lpa2-a mutations (Gillman, et al. (2009)), one would expect that the loss of function mutation lpa2-b in M766 would result in the reduction of the phytic acid transporter activity below the threshold necessary to produce a measurable phosphorus partitioning phenotype, even in the presence of functional versions of Lpa1. Surprisingly, as demonstrated herein, the lpa2-b mutation alone is not sufficient to generate a mutant soybean line with extremely low phytic acid and high inorganic phosphate levels in the seeds. After determining the genotype of the F₂ seed of the M766×‘Williams 82’, the seeds are examined for the phenotypic consequences of presence or absence of alleles derived from M766 on seed phosphorus partitioning by determining the inorganic phosphate (Pi) content of 10-30 μg of seed tissue using the protocol described in Gillman, et al. (2009) and in Wilcox, et al. (2000). It is assumed that ‘Williams 82’ contains functional alleles of Lpa1 and Lpa2. Only homozygosity for both the lpa1-b and lpa2-b alleles is found to be associated with significantly elevated levels of inorganic phosphate compared to the ‘Williams 82’ parent for the F₂ seeds (see FIG. 2). This finding is consistent with the previous findings for CX1834 (see Gillman, et al. (2009)). The necessity for the combination of homozygous lpa1-b and lpa2-b alleles for the high inorganic phosphate phenotype is confirmed by examining the inorganic phosphate content of F₃ progeny of an F₂ selection which is homozygous for the lpa2-b allele, but was heterozygous for Lpa1 using the previously described assay (see FIG. 3). F₃ seeds containing the lpa2-b alleles and one or two functional Lpa1 alleles possess available phosphate levels just above the detection limit of the assay. Seeds homozygous for the combination of the lpa2-b alleles and the lpa1-b alleles had approximately seven-fold more available phosphate, close to the level achieved by M766 (see FIG. 3 and FIG. 7).

Linkage of the lpa2-b allele with altered phosphorus partitioning suggests it is not equivalent to a fully functional allele. To fully elucidate this hypothesis, DNA is isolated from ˜20 μg of F₃ seed tissue using a DNeasy kit (Qiagen, Inc., Valencia, Calif.) and is sequenced. No changes in the coding sequence or alterations to the promoter (within 240 basepairs upstream) are detected in the lpa1-b allele from M766. A single T>A SNP is identified seven base pairs upstream of the start of exon 10. Based on software prediction analysis of this SNP (http://www.cbs.dtu.dk/servoces/NetPGene/), it is hypothesized that a cryptic splice site is activated resulting in the introduction of five additional base pairs of intron sequence and a frame shift mutation starting at exon 10.

To confirm this hypothesis, mRNA of pods corresponding to two stages of seed fill (8-10 mm in length and >10 mm/fully expanded seeds) are collected from field grown plants of ‘Williams 82’, ‘M766’, and the F_{2:3} line which recreated the lpa1-b/lpa2-b homozygote mutant allele combination (line 6-8-31). Seeds are separated from pods before being flash frozen and ground in liquid nitrogen. mRNA is extracted from ˜50 mg seed powder using the Trizol reagent (Invitrogen, Carlsbad, Calif.) according to manufacturer's instructions. The mRNA is purified and on-column DNase treated using the Direct-Zol RNA miniprep kit, according to manufacturer's recommendations (Zymo, Irvine, Calif.). 0.4 μg of DNase treated mRNA is used with the SMARTScribe Reverse Transcriptase kit with a gene specific primer located in exon 10 of Lpa1 (5′-GGAGGGCGAGAATCTTCAATAAT-3′ (SEQ ID NO:4)) according to manufacturer's recommendations (Clontech, Mountain View, Calif.), and RT-PCR is carried out using a SYBR green Quantitect PCR kit according to manufacturer's recommendations (Qiagen, Germantown, Md.), using a forward primer located in exon 9 (5′-TATTGTTAGCATACAGAAGTCTCCAAT-3′ (SEQ ID NO:5)) and a reverse primer located in exon 10 of Lpa1 (5′-GGAGGGCGAGAATCTTCAATAAT-3′ (SEQ ID NO:6)) primers. RT-PCR products are verified to be of appropriate size, are purified using a Qiaquick PCR purification kit (Qiagen, Germantown, Md.), and are Sanger sequenced at the DNAcore facility at the University of Missouri. The resulting sequence traces are imported into the Geneious software package (v5.6) (Auckland, New Zealand) and are manually trimmed and aligned. To verify the putative mixture of spliced and mispliced transcripts, a fluorescently labeled version of the forward primer (TAMRA 5′-TATTGTTAGCATACAGAAGTCTCCAAT-3′ (SEQ ID NO:7)) is used in RT-PCR, with amplification products purified as described previously, and are diluted 1:15 and 1:50 fold before submission to the DNAcore facility at the University of Missouri for size fractionation. The exact sizes of RT-PCR products are determined by comparison to a standard mixture of size standards using the PeakScanner software package (Applied Biosystems, Carlsbad, Calif.).

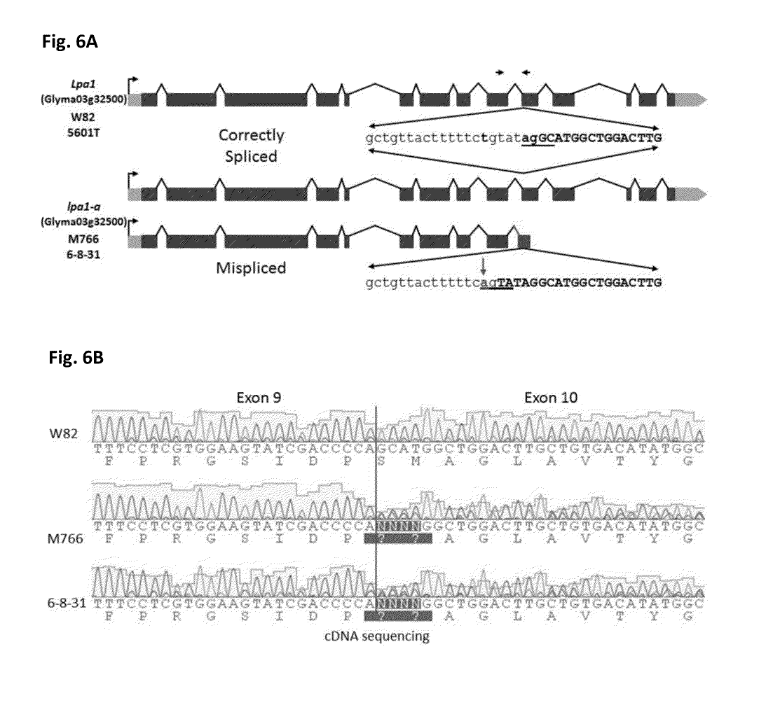

These assays confirm that a portion of transcripts from the lpa1-b locus are mispliced (see FIG. 6A and FIG. 6B). The cryptic splice site is activated in only a subset of the total lpa1-b transcripts which likely explains the finding that the lpa1-b allele is inferior as a means of reducing the anti-nutritional compound phytic acid as compared to the nonsense lpa1-a mutation found in M153/CX1834 (Gillman, et al. (2009)).

### EXAMPLE 3

### Phosphate Partitioning in F₂ Seeds from M766×TN09-239

Although the combination of alleles found in M766 (lpa1-b, lpa2-b) is unlikely to be useful in breeding efforts for reducing seed phytic acid (PA), the combination of the lpa2-b nonsense mutation with the lpa1-a nonsense mutation is examined to determine if the combination could increase inorganic phosphate (Pi) to a greater extent than possible in the prior art CX1834 or derived lines (Gillman, et al. (2009)).

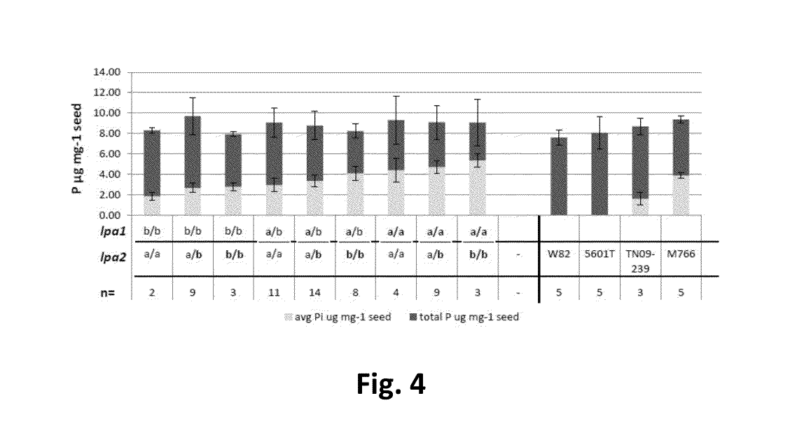

As described in Example 1, a population of plants is developed from the cross of TN09239×M766, and the F₂ genotypes are examined. F₂ seeds, produced in a growth chamber environment as describe above, are analyzed for inorganic phosphate levels using the protocols set for in Gillman, et al. (2009) and Wilcox, et al. (2000). Data from individual seeds are grouped into appropriate genotypic classes for statistical analysis (see FIG. 4). For the two wild type soybean lines ‘Williams 82’ (W82) and 5601T, inorganic phosphate is virtually undetectable in seeds. In contrast, both TN09-239 (bearing homozygous mutant alleles lpa1-a and lpa2-a) and M766 (bearing homozygous alleles of lpa1-b and the nonsense mutant alleles of lpa2-b) possess elevated inorganic phosphate: 1.63±0.60 μg P mg⁻¹ seed and 3.90±0.24 μg P mg⁻¹seed, respectively. Curiously, the seeds produced by TN09-239 line in the growth chamber possess a lower amount of inorganic phosphate as compared to M766 as well as slightly lower levels of total seed phosphorus (although this finding is not statistically significant) as compared to M766 seeds (FIG. 4). This result is unexpected and may result from growing a maturity group V line in an artificial environment optimized for the maturity group III lines M766 and ‘Williams 82’.

The progeny M766×TN09-239 are assessed for the phenotypic consequences of the combinations of the different mutant versions of lpa1 and lpa2. It was previously hypothesized (Gillman, et al. (2009)) that the missense lpa2-a allele found in M153/CX1834 might only impair functionality, rather than abolishing it. If this hypothesis is correct, then the combination of two nonsense mutations affecting the two independently inherited paralogous genes (lpa1-a derived from M153/CX1834 in conjunction with lpa2-b from M766) could result in higher levels of available inorganic phosphate in seeds. Inorganic phosphate content of the seeds is quantified using between 10 μg and 30 μg of seed tissue using the protocol set forth in Gillman, et al. (2009) and in Willcox, et al. (2000). The combination of the lpa1-b allele from M766 with the lpa2-a allele from CX1834 (lpa1-b/lpa2-a) results in accumulation of the least available inorganic phosphate (1.88±0.42 μg P mg⁻¹ seed) amongst the mutant genotypic classes, and the novel homozygote combination of the two nonsense mutant alleles, lpa1-a/lpa2-b, surprisingly produces the highest available inorganic phosphate of any of the samples examined (5.39±0.65 μg P mg⁻¹ seed) (see FIG. 4). Previous examples with different germplasm sources of the low phytate phenotype have consistently shown strong negative correlations between the amount of phosphorous measured in available phosphate and the amount of phosphorous measured in phytate (Bilyeu, et al., Plant Physiology 146:468-477 (2008); Gillman, et al. (2009)), so these F₂ experiments do not include phytate measurements.

### EXAMPLE 4

### Analysis of F₃ Seeds from F₂ Genotypic Field Grown Selections

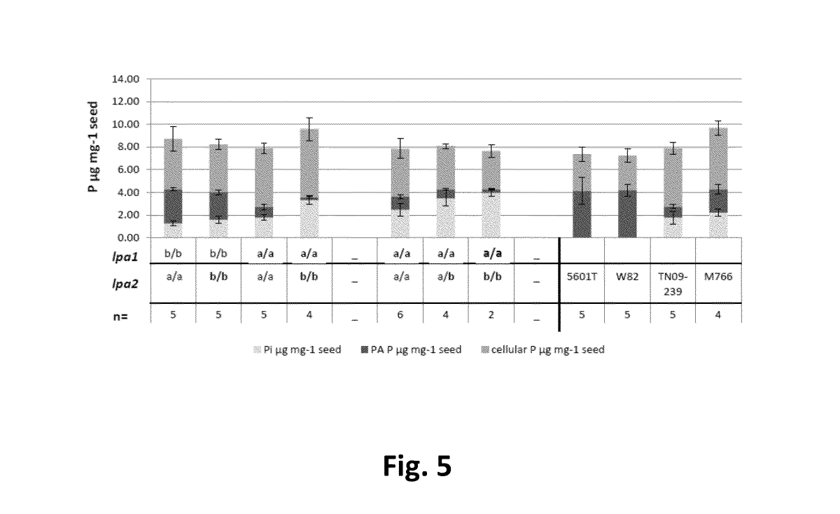

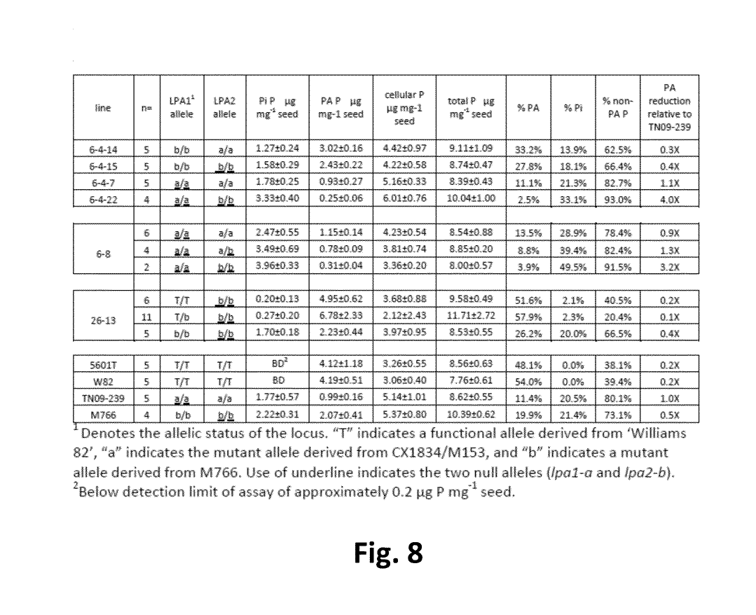

More comprehensive analyses of inorganic phosphate and phytic acid levels are carried out on field produced F_{2:3} seeds derived from remnant F₂ seeds from the populations. Plants representing all of the possible homozygous genotypic combinations of lpa1 and lpa2 mutations (lpa1-a/lpa2-a, lpa1-a/lpa2-b, lpa1-b/lpa2-a, lpa1-b/lpa2-b) are selected (after determining genotypes as described in Example 1) and four or five F₃ seeds from each plant are analyzed independently to investigate three distinct seed phosphorus related traits: available inorganic phosphate by colorimetric analysis, phytic acid content by HPLC analysis, and total cellular phosphorus as determined by ICP-MS (see FIG. 5 and FIG. 8). Inorganic phosphate content is measured as per the protocol set forth in Gillman, et al. (2009) and Wilcox, et al. (2000).

Phytic acid (PA) content of the F₃ soybean seeds is quantified by modified HPLC method (Chen and Li (2003)). Powdered seed samples (0.025 g) are extracted by shaking at room temperature in 0.5 mL of 0.5 N HCl for one hour. After centrifugation for 15 minutes at 15,000×g, supernatants are filtered through a 0.22-μ filter, and 100 μl of filtrate are analyzed. PA and inositol polyphosphate separations are performed by a linear gradient elution program on a Dionex CarboPac PA-100 guard column and a CarboPac PA-100 analytical column on a Dionex ICS-5000 Ion Chromatography System (Thermo Scientific, Waltham, Mass.). The elution gradient is effected by a mixture of two eluents: water and 0.5 N HCl; time 0 minutes, 8% 0.5 N HCl; time 30 minutes, 92% 0.5 N HCl; time 35 minutes, 92% 0.5 N HCl; time 35.1 minutes, 8% 0.5 N HCl; time 40 minutes, stop run. A post-column derivitization is achieved with a solution of 1 g/L Fe(NO₃)₃ in 0.33 M HClO₄ using a 750-μL knitted coil and is followed by detection of A₂₉₅. Flow rates of eluent and post-column solution are 1.0 and 0.4 ml/minute, respectively. PA standard (phytic acid dipotassium salt; Sigma Aldrich, St. Louis, Mo.) elutes at 30 minutes. Standard curves are calculated from dilutions of PA standards. Results are converted to μg phytic acid P per mg seed.

Quantification of soybean seed total ionome is determined as follows. Powdered seed samples are aliquoted into 16×100 mm Pyrex test tubes and are weighed. Digestion is carried out by adding 2.5 ml of concentrated HNO₃ (VWR AR Select ACS grade, Radnor, Pa.) containing Indium internal standard to the test tubes and incubating overnight at room temperature before heating the samples to 105° C. over two hours and then cooling to room temperature over two hours. The digested samples are diluted in the test tubes to 10 ml by adding ultrapure water, and a second dilution is made in a second set of test tubes by taking 900 μl of the first dilutions to 5 ml with ultrapure water. Then 1.2 ml of the second dilutions are transferred to 96-well autosampler plates using an adjustable-width multi-channel pipette. Elemental analysis is performed using an ICP-MS (Perkin-Elmer Elan DRCe; Waltham, Mass.) with an Apex Desolvation Nebulizer, FAST sampling valve, and SC4 DX autosampler (Elemental Scientific, Inc., Omaha, Nebr.). A liquid reference material composed of pooled samples of soybean digests is run every 9^{th} sample to correct for ICPMS within-run drift. All samples are normalized to the recorded weights.

Similar to the situation for growth chamber produced seeds, free inorganic phosphate is present in trace, nearly undetectable amounts for seeds from field grown wild type soybean lines (see FIGS. 7 and 8). All homozygote combinations of mutant alleles for lpa1 and lpa2 result in elevation of inorganic phosphate and a concomitant reduction in phytic acid (FIG. 5). However, the novel combination of the lpa1-a mutation from CX1834 with the lpa2-b mutation from M766 surprisingly results in the greatest accumulation of available inorganic phosphate phosphorus (3.33±0.40 μg P mg⁻¹ seed) and the least amount of phytic acid phosphorus (0.25±0.06 μg P mg⁻¹ seed). The amount of available inorganic phosphate (Pi) is approximately 1.88× higher than noted for TN09-239 (1.77±0.57 μg P mg⁻¹ seed) or the F₂ selection homozygous for lpa1-a/lpa2-a (1.78±0.25 μg P mg⁻¹ seed), and these differences are statistically significant (p=0.0024). In addition, phytic acid phosphorus for the lpa1-a1 lpa2-b line is surprisingly and dramatically reduced approximately 4-fold for the novel allele combination (0.25±0.06 μg P mg⁻¹ seed) as compared to TN09-239 (0.99±0.16 μg P mg⁻¹ seed) or when compared to the F₃ selection bearing lpa1-a/lpa2-a (0.90±0.19, approximately 3.6-fold reduction), and these differences are statistically significant (p<0.0001). In comparison to the wild-type line 5601T (4.12±1.18 μg P mg⁻¹ seed), the reduction in phytic acid in the lpa1-a/lpa2-b line is approximately 16.5-fold.

### EXAMPLE 5

### Analysis Phosphorus Partition in F₃ Progeny of F₂ Selections Heterozygous for One lpa Locus

The phosphorus partitioning in seeds for the progeny of a plant heterozygous for lpa1-a, but homozygous for lpab2-b, is performed using the above described protocols. The results confirm the unexpected findings described above (see FIG. 5) that the novel combination lpa1-a/lpab2-b results in greater reductions in phytic acid than is possible using M153/CX1834 derived alleles alone, as evinced by TN09-239. A concomitant increase in the available inorganic phosphate (Pi) is also noted in the novel lpa1-a/lpab2-b line. Amongst these selections, there is no significant difference in terms of total phosphorus, as detected by ICP-MS (see FIGS. 7 and 8). The percentage of potentially available phosphorus (total P-phytic acid P) ranges from 42.2-44.2% for wild type samples (Williams 82 and 5601T, respectively) to 87.5% for TN09-239 or the genotypic class representing lpa1-a/lpa2-a (88.5%). The novel combination of lpa1-a/lpa2-b results in a remarkable approximately 97.4% of the total phosphorus potentially available for digestion, with only a very minute trace amount of indigestible phytic acid P present (0.25±0.06 μg PA P mg⁻¹ seed, approximately 2.6% of total phosphorus).

### EXAMPLE 6

### Analysis of the Impact of Low Phytic Acid Alleles on Amount of Other Minerals in the Seeds

ICP-MS analysis of the seeds in Example 5 above yields a considerable amount of data regarding the mineral composition of tissues. Notable differences exist for certain mineral species between parental genotypes. For example, ‘Williams 82’ possesses a low level of nickel, as compared to plants of the other genotypes grown in the same environment. Similarly, both ‘Williams 82’ and TN09-239 have lower levels of copper as compared to 5601T and M766. M766 also has significantly higher levels of total phosphorus content. The genetic factors responsible and/or biological significance (if any) of these mineral compositional differences is unknown at this time.

In contrast to the results noted for the parental lines, there does not seem to be any consistent, statistically significant differences among any of the lpa1/2 genotypic classes for F₂ or F₃ generations in terms of elemental mineral content. However, soymeal derived from plants bearing the novel lpa1-a/lpa2-b allele combination may have much higher levels of zinc and iron available for uptake. Not wishing to be bound to any particular theory, it is believed that the dramatic reduction in phytic acid content in the line results in very little chelating of minerals by phytic acid.

### EXAMPLE 7

### Seedling Emergence

In order to evaluate any large effect issues with seedling emergence, a small scale field study with F₂ seeds is performed. All genotypic classes for both populations from seeds produced in a growth chamber are able to germinate, emerge, and produce seed at the Bradford Research and Extension Center near Columbia, Mo. during the summer. No significant segregation distortion at F₂ or F₃ for the cross of M766×TN09-239 (FIG. 7) is observed. Lines with all combinations of mutant lpa1/lpa2 alleles are capable of germination and field emergence. Segregation distortion is observed in the cross between M766×W82, with an overrepresentation of wild type samples occurring for LPA2 (FIG. 7), similar to studies previously reported for low phytic acid wheat lines (Guttieri, et al., Crop Science 44:418-424 (2004)).

Having now generally described this invention, the same will be better understood by reference to certain specific examples and the accompanying drawings, which are included herein only to further illustrate the invention and are not intended to limit the scope of the invention as defined by the claims. The examples and drawings describe at least one, but not all embodiments, of the inventions claimed. Indeed, these inventions may be embodied in many different forms and should not be construed as limited to the embodiments set forth herein; rather, these embodiments are provided so that this disclosure will satisfy applicable legal requirements.

We, the inventors, claim:

1. A plant comprising alleles lpa1-a and lpab2-b wherein the seeds of said plant has reduced seed phytate levels and higher inorganic phosphate levels compared to seeds of wild-type plant.

2. The plant of claim 1 wherein said plant is Glycine max.

3. The seeds of said plant of claim 1.

4. A cell of said plant of claim 1.

5. Germplasm which will yield the seeds of claim 3.

6. A plant comprising mutant alleles affecting Lpa1 and Lpa2 genes wherein the seeds of said plant has reduced seed phytate levels and higher inorganic phosphate levels compared to seeds of plants containing the lpa1-a and lpa2-a mutant alleles.

7. The plant of claim 6 wherein said plant is Glycine max.

8. The seeds of said plant of claim 6.

9. Germplasm which will yield the seeds of claim 8.

10. A cell of said plant of claim 6.

11. A seed of a soybean plant having two non-lethal mutations wherein said seed is characterized by lower phytic acid levels and higher inorganic phosphorus levels compared to soybean plant cultivar TN09-239.

12. The seed of claim 11 wherein said phenotype is caused by mutations in Lpa1 and Lpa2 genes.

13. A soybean plant produced by the seed of claim 11.

14. Germplasm that will yield a seed of claim 11.
